(19) United States
(12) Patent Application Publication
(10) Pub. No.: US 2022/0257327 A1
(43) Pub. Date: Aug. 18, 2022

(54) WRIST ASSEMBLY FOR ROBOTIC INSTRUMENTS

(71) Applicant: Covidien LP, Mansfield, MA (US)

(72) Inventor: Jaroslaw T. Malkowski, North Port, FL (US)

(21) Appl. No.: 17/618,907

(22) PCT Filed: Aug. 26, 2020
(86) PCT No.: PCT/US2020/047864

§ 371 Date: Dec. 14, 2021

Related U.S. Application Data
(60) Provisional application No. 62/894,278, filed on Aug. 30, 2019.

(51) Int. Cl. A61B 34/00; A61B 34/37

(52) U.S. Cl. CPC A61B 34/71; A61B 2017/00314; A61B 34/37

(57) ABSTRACT

A wrist assembly for a surgical instrument includes a proximal joint member, a middle joint member, and a distal joint member. The proximal joint member defines a concavity formed in a distal facing surface thereof. The distal joint member defines concavity formed in a proximal facing surface thereof, a longitudinally extending central lumen, and a plurality of longitudinally extending radial lumens arranged in a radial array around the central lumen thereof. The middle joint member is interposed between the proximal joint member and the distal joint member. The middle joint member includes a proximal ball connector extending proximally therefrom and a distal ball connector extending distally therefrom, wherein the proximal ball connector is seated within the concavity formed in the distal facing surface of the proximal joint member and the distal ball connector is seated within the concavity formed in the proximal facing surface of the proximal joint member.

### BACKGROUND

Robotic surgical systems have been used in minimally invasive medical procedures. Some robotic surgical systems include a console supporting a robot arm, and at least one end effector such as forceps or a grasping tool that is mounted to the robot arm via a wrist assembly. During a medical procedure, the end effector and the wrist assembly are inserted into a small incision (via a cannula) or a natural orifice of a patient to position the end effector at a work site within the body of the patient.

In robotic surgical systems, cables extend from the robot console, through the robot arm, and connect to the wrist assembly and/or end effector. In some instances, the cables are actuated by means of motors that are controlled by a processing system including a user interface for a surgeon or clinician to be able to control the robotic surgical system including the robot arm, the wrist assembly and/or the end effector.

Existing wristed robotic instruments also have what is known as an elbowed design in which the end effector articulation point and the jaw pivot point are located at longitudinally spaced apart positions. Consequently, given the variety of positions in which these end effectors may be disposed to access surgical sites, one challenge associated with existing wristed robotic instruments is providing increased ranges of motions, access and reach to the wristed robotic instruments so as to enable a surgeon to perform greater numbers of surgical procedures.

Accordingly, a need exists for wristed robotic instruments that provide increased ranges of motions, access and reach which provide a surgeon with increased ability to perform greater numbers of surgical procedures.

### SUMMARY

Accordingly, this disclosure details mechanical implementations for wristed robotic instruments that provide increased ranges of motions, access and reach to a surgeon, thereby enabling the surgeon to perform greater numbers of surgical procedures.

In accordance with one aspect, this disclosure is directed to a surgical instrument for a robotic surgical system. The surgical instrument includes an elongate shaft defining a longitudinal axis; an end effector having a first jaw member and a second jaw member movably coupled to one another; and a wrist assembly interconnecting the elongate shaft and the end effector, the wrist assembly permitting a longitudinal axis of the end effector to be rotated or translated relative to the longitudinal axis of the elongate shaft.

The wrist assembly includes a proximal joint member supported on a distal end of the elongate shaft. The proximal joint member defines a longitudinally extending central lumen; a plurality of longitudinally extending radial lumens arranged in a radial array around the central lumen thereof; and a concavity formed in a distal facing surface thereof, wherein the concavity of the proximal joint member is centrally aligned with the central lumen thereof.

The wrist assembly includes a distal joint member supported on a proximal end of the end effector. The distal joint member defines a longitudinally extending central lumen; a plurality of longitudinally extending radial lumens arranged in a radial array around the central lumen thereof; and a concavity formed in a proximal facing surface thereof, wherein the concavity of the distal joint member is centrally aligned with the central lumen thereof.

The wrist assembly includes a middle joint member interposed between the proximal joint member and the distal joint member. The middle joint member includes a proximal ball connector extending proximally therefrom and a distal ball connector extending distally therefrom, wherein the proximal ball connector is seated within the concavity formed in the distal facing surface of the proximal joint member and the distal ball connector is seated within the concavity formed in the proximal facing surface of the proximal joint member. The distal joint member defines a longitudinally extending central lumen; and a plurality of longitudinally extending radial lumens arranged in a radial array around the central lumen thereof.

The wrist assembly may include a proximal bearing disposed within the concavity of the proximal joint member, and interposed between the proximal joint member and the proximal ball connector of the middle joint member; and a distal bearing disposed within the concavity of the distal joint member, and interposed between the distal joint member and the distal ball connector of the middle joint member.

The proximal bearing may define a pair of diametrically opposed, radially outwardly projecting bosses that define a pivot axis therethrough; and the distal bearing may define a pair of diametrically opposed, radially outwardly projecting bosses that define a pivot axis therethrough.

The pivot axis of the pair of bosses of the proximal bearing and the pivot axis of the pair of bosses of the distal bearing may reside in a common plane.

The proximal joint member may define a pair of diametrically opposed, radially outward projecting pivot grooves formed in a rim of the concavity thereof and may be configured and dimensioned to pivotably receive the pair of bosses of the proximal bearing. The distal joint member may define a pair of diametrically opposed, radially outward projecting pivot grooves formed in a rim of the concavity thereof and may be configured and dimensioned to pivotably and slidably receive the pair of bosses of the distal bearing.

The proximal ball connector of the middle joint member may define a pair of diametrically opposed, radially outwardly projecting bosses that define a pivot axis therethrough. The distal ball connector of the middle joint member may define a pair of diametrically opposed, radially outwardly projecting bosses that define a pivot axis therethrough.

The pivot axis of the pair of bosses of the proximal ball connector and the pivot axis of the pair of bosses of the distal ball connector may reside in a common plane.

The pivot axis of the pair of bosses of the proximal bearing may be orthogonal to the pivot axis of the pair of bosses of the proximal ball connector.

The pivot axis of the pair of bosses of the distal bearing may be orthogonal to the pivot axis of the pair of bosses of the distal ball connector.

The proximal bearing may defines a pair of diametrically opposed, radially inward projecting pivot grooves formed in a rim of a concavity thereof and which grooves may be configured and dimensioned to pivotably and slidably receive the pair of bosses of the proximal ball connector of the middle joint member. The distal bearing may define a pair of diametrically opposed, radially inward projecting pivot grooves formed in a rim of a concavity thereof and which grooves may be configured and dimensioned to pivotably and slidably receive the pair of bosses of the distal ball connector of the middle joint member.

The distal joint member may include four longitudinally extending radial lumens arranged in a radial array around the central lumen thereof. The middle joint member may include eight longitudinally extending radial lumens arranged in a radial array around the central lumen thereof, wherein four of the eight radial lumens of the middle joint member may be in registration with the four radial lumens of the distal joint member.

The proximal joint member may include eight longitudinally extending radial lumens arranged in a radial array around the central lumen thereof, wherein the eight radial lumens of the proximal joint member are in registration with the eight radial lumens of the middle joint member.

The surgical instrument may further include a plurality of articulation cables extending through the wrist assembly. In an embodiment, four articulation cables of the plurality of cables may include distal ends secured to the distal joint member, and proximal ends slidably extending through the four longitudinally extending radial lumens of the middle joint member that are in registration with the four longitudinally extending radial lumens of the distal joint member. The four articulation cables of the plurality of cables may include distal ends secured to the middle member, and proximal ends slidably extending through the four longitudinally extending radial lumens of the proximal joint member that are in registration with the four un-occupied longitudinally extending radial lumens of the middle joint member.

The proximal bearing and the distal bearing may be fabricated from a lubricious material.

According to another aspect of the disclosure, a wrist assembly for a surgical instrument is provided. The wrist assembly includes a proximal joint member configured for support on a distal end of an elongate shaft, a distal joint member configured to support a proximal end of a surgical end effector, and a middle joint member interposed between the proximal joint member and the distal joint member. The proximal joint member defines a longitudinally extending central lumen; a plurality of longitudinally extending radial lumens arranged in a radial array around the central lumen thereof; and a concavity formed in a distal facing surface thereof, wherein the concavity of the proximal joint member is centrally aligned with the central lumen thereof.

The distal joint member defines a longitudinally extending central lumen; a plurality of longitudinally extending radial lumens arranged in a radial array around the central lumen thereof; and a concavity formed in a proximal facing surface thereof, wherein the concavity of the distal joint member is centrally aligned with the central lumen thereof.

The middle joint member includes a proximal ball connector extending proximally therefrom and a distal ball connector extending distally therefrom, wherein the proximal ball connector is seated within the concavity formed in the distal facing surface of the proximal joint member and the distal ball connector is seated within the concavity formed in the proximal facing surface of the proximal joint member.

The distal joint member defines a longitudinally extending central lumen; and a plurality of longitudinally extending radial lumens arranged in a radial array around the central lumen thereof.

The wrist assembly may further include a proximal bearing disposed within the concavity of the proximal joint member, and interposed between the proximal joint member and the proximal ball connector of the middle joint member; and a distal bearing disposed within the concavity of the distal joint member, and interposed between the distal joint member and the distal ball connector of the middle joint member.

The proximal bearing may define a pair of diametrically opposed, radially outwardly projecting bosses that define a pivot axis therethrough; and the distal bearing may define a pair of diametrically opposed, radially outwardly projecting bosses that define a pivot axis therethrough.

The pivot axis of the pair of bosses of the proximal bearing and the pivot axis of the pair of bosses of the distal bearing may reside in a common plane.

The proximal joint member may define a pair of diametrically opposed, radially outward projecting pivot grooves formed in a rim of the concavity thereof and may be configured and dimensioned to pivotably receive the pair of bosses of the proximal bearing. The distal joint member may define a pair of diametrically opposed, radially outward projecting pivot grooves formed in a rim of the concavity thereof and may be configured and dimensioned to pivotably and slidably receive the pair of bosses of the distal bearing.

The proximal ball connector of the middle joint member may define a pair of diametrically opposed, radially outwardly projecting bosses that define a pivot axis therethrough. The distal ball connector of the middle joint member may define a pair of diametrically opposed, radially outwardly projecting bosses that define a pivot axis therethrough.

The pivot axis of the pair of bosses of the proximal ball connector and the pivot axis of the pair of bosses of the distal ball connector may reside in a common plane.

The pivot axis of the pair of bosses of the proximal bearing may be orthogonal to the pivot axis of the pair of bosses of the proximal ball connector.

The pivot axis of the pair of bosses of the distal bearing may be orthogonal to the pivot axis of the pair of bosses of the distal ball connector.

The proximal bearing may define a pair of diametrically opposed, radially inward projecting pivot grooves formed in a rim of a concavity thereof and which grooves are configured and dimensioned to pivotably and slidably receive the pair of bosses of the proximal ball connector of the middle joint member. The distal bearing may define a pair of diametrically opposed, radially inward projecting pivot grooves formed in a rim of a concavity thereof and which grooves are configured and dimensioned to pivotably and slidably receive the pair of bosses of the distal ball connector of the middle joint member.

The distal joint member may include four longitudinally extending radial lumens arranged in a radial array around the central lumen thereof. The middle joint member may include eight longitudinally extending radial lumens arranged in a radial array around the central lumen thereof, wherein four of the eight radial lumens of the middle joint member are in registration with the four radial lumens of the distal joint member.

The proximal joint member may include eight longitudinally extending radial lumens arranged in a radial array around the central lumen thereof, wherein the eight radial lumens of the proximal joint member are in registration with the eight radial lumens of the middle joint member.

The wrist assembly may further include a plurality of articulation cables extending therethrough. In an embodiment, four articulation cables of the plurality of cables include distal ends secured to the distal joint member, and proximal ends slidably extending through the four longitudinally extending radial lumens of the middle joint member that are in registration with the four longitudinally extending radial lumens of the distal joint member. The four articulation cables of the plurality of cables may include distal ends secured to the middle member, and proximal ends slidably extending through the four longitudinally extending radial lumens of the proximal joint member that are in registration with the four un-occupied longitudinally extending radial lumens of the middle joint member.

The proximal bearing and the distal bearing may be fabricated from a lubricious material.

Other aspects, features, and advantages will be apparent from the description, the drawings, and the claims that follow.

### BRIEF DESCRIPTION OF DRAWINGS

The accompanying drawings, which are incorporated in and constitute a part of this specification, illustrate embodiments of the disclosure and, together with a general description of the disclosure given above and the detailed description given below, serve to explain the principles of the disclosure, wherein:

FIG. 1 is a perspective view of a robotic surgical system in accordance with this disclosure;

FIG. 2 is an enlarged schematic illustration of the indicated area of detail of FIG. 1, illustrating a distal end of a surgical instrument of the robotic surgical system shown in an articulated condition;

FIG. 3 is an enlarged rear perspective view of a wrist assembly of the surgical instrument shown in FIGS. 1 and 2;

FIG. 4 is a perspective view, with parts separated, of the wrist assembly shown in FIG. 3;

FIG. 5 is a longitudinal, cross-sectional view of the wrist assembly shown in FIG. 3;

FIG. 5 is a perspective view, with parts separated, of the wrist assembly of FIGS. 2-4;

FIG. 6 is an enlarged front perspective view of the wrist assembly shown in FIG. 3;

FIG. 7 is a cross-sectional view of the wrist assembly shown in FIG. 6, as taken through 7-7 of FIG. 6;

FIG. 8 is a perspective view of the wrist assembly shown in FIG. 7, illustrating an articulation of a distal joint member relative to a middle joint member;

FIG. 9 is a cross-sectional view of the wrist assembly shown in FIG. 6, as taken through 9-9 of FIG. 6;

FIG. 10 is a perspective view of the wrist assembly shown in FIG. 9, illustrating an articulation of a distal joint member relative to a middle joint member;

FIG. 11 is an enlarged perspective view of a wrist assembly for a surgical instrument in accordance with another embodiment of the present disclosure;

FIG. 12 is a perspective view, with parts separated, of the wrist assembly shown in FIG. 11.

FIG. 13 is a longitudinal cross-sectional view of the wrist assembly of FIGS. 11-12, while the wrist assembly is in a non-articulated condition;

FIG. 14 is a longitudinal, cross-sectional view of a proximal joint member of the wrist assembly shown in FIG. 12; and

FIG. 15 is a perspective view of the wrist assembly shown in FIG. 11, as taken through 15-15 of FIG. 11.

### DETAILED DESCRIPTION

Embodiments of the present disclosure are described in detail with reference to the drawings, in which like reference numerals designate identical or corresponding elements in each of the several views. As commonly known, the term “clinician” refers to a doctor, surgeon, a nurse, or any other care provider and may include support personnel. Additionally, the term “proximal” refers to the portion of structure that is closer to the clinician and the term “distal” refers to the portion of structure that is farther from the clinician. In addition, directional terms such as front, rear, upper, lower, top, bottom, and the like are used simply for convenience of description and are not intended to limit the disclosure attached hereto.

In the following description, well-known functions or constructions are not described in detail to avoid obscuring the present disclosure in unnecessary detail.

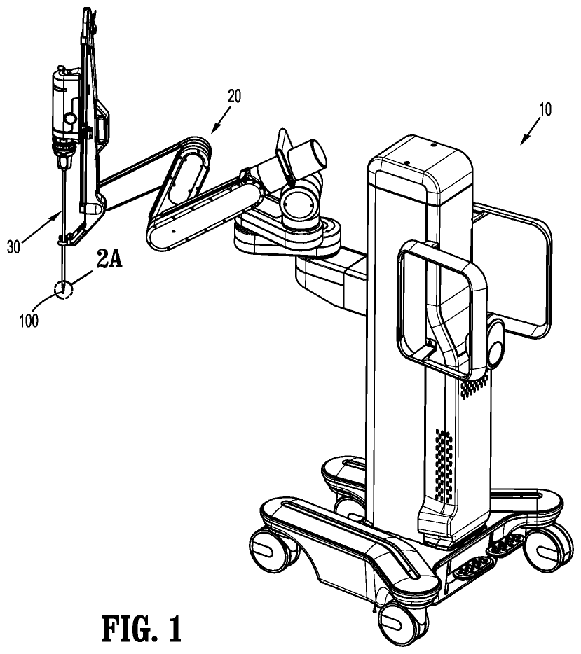

With brief reference to FIG. 1, a robotic surgical system 10 is shown and includes a robotic arm 20 that supports a wristed surgical instrument 30 having an end effector 100. Robotic surgical system 10 employs various robotic elements to assist the clinician and allow remote operation (or partial remote operation) of surgical instrumentation such as surgical instrument 30. Various robotic arms, gears, cams, pulleys, electric and mechanical motors, etc. may be employed for this purpose and may be designed with robotic surgical system 10 to assist the clinician during the course of an operation or treatment.

Robotic surgical system 10 may be employed with one or more consoles (not shown) that are next to the operating theater or located in a remote location. In this instance, one team of clinicians may prep the patient for surgery and configure the robotic surgical system with one or more of the instruments/end effectors disclosed herein while another clinician (or group of clinicians) remotely controls the instruments/end effectors via robotic surgical system 10. As can be appreciated, a highly skilled clinician may perform multiple operations in multiple locations without leaving his/her remote console which can be both economically advantageous and a benefit to the patient or a series of patients. For a detailed description of exemplary medical work stations and/or components thereof, reference may be made to U.S. Patent Application Publication No. 2012/0116416 and PCT Application Publication No. WO2016/025132, the entire contents of each of which are incorporated by reference herein.

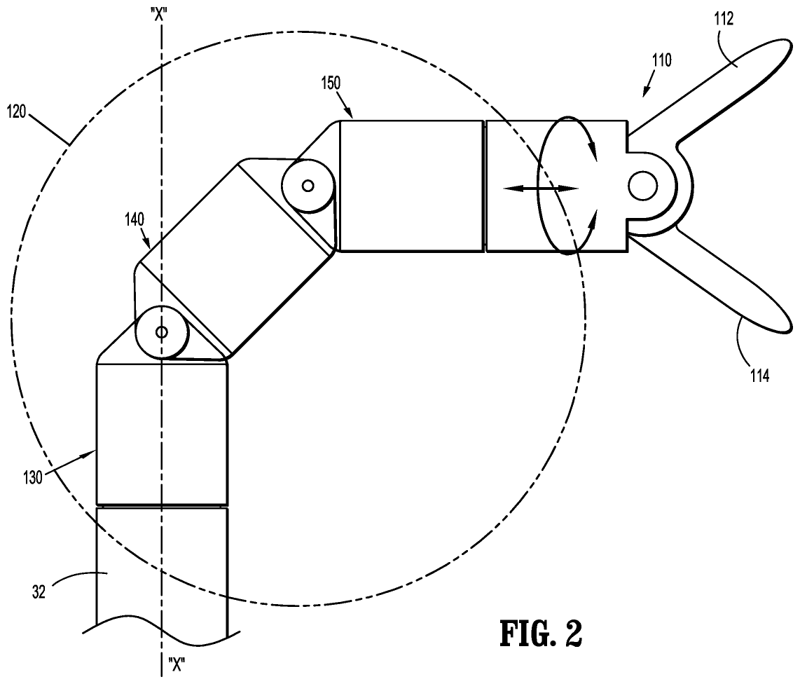

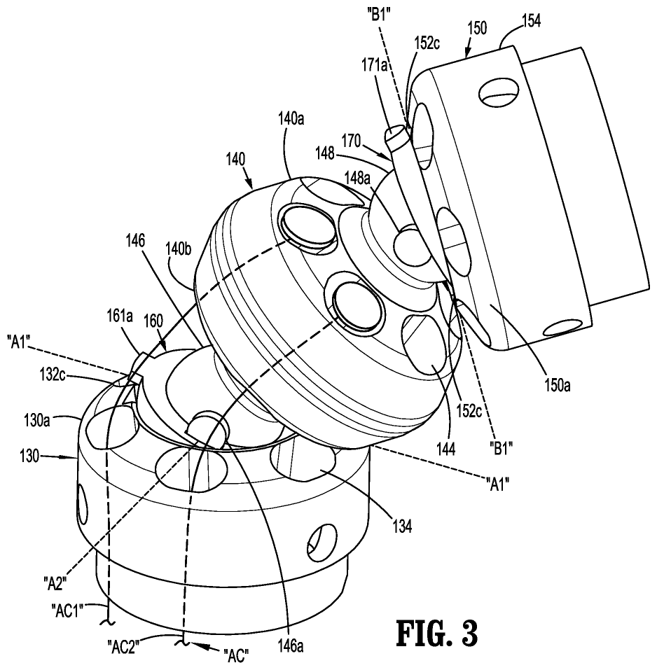

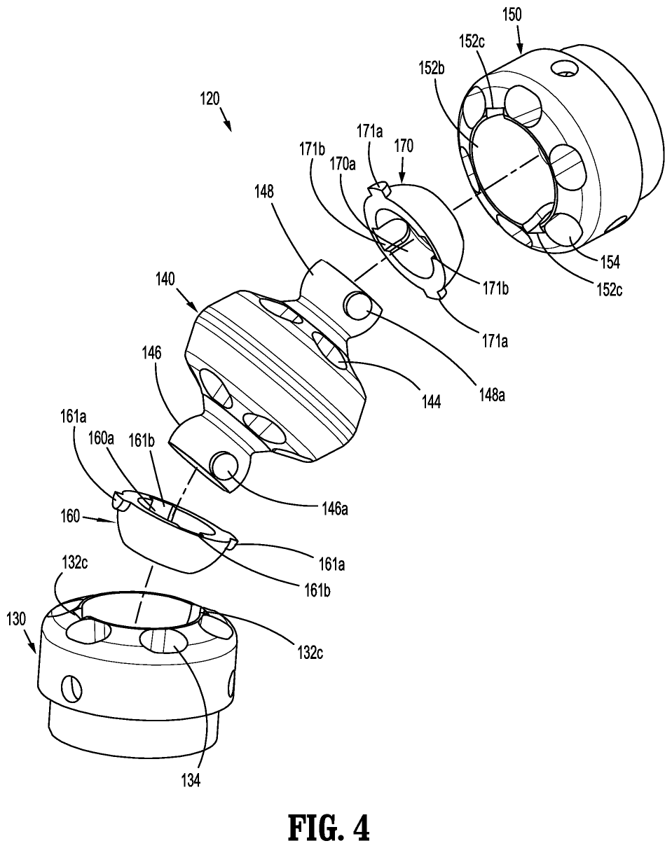

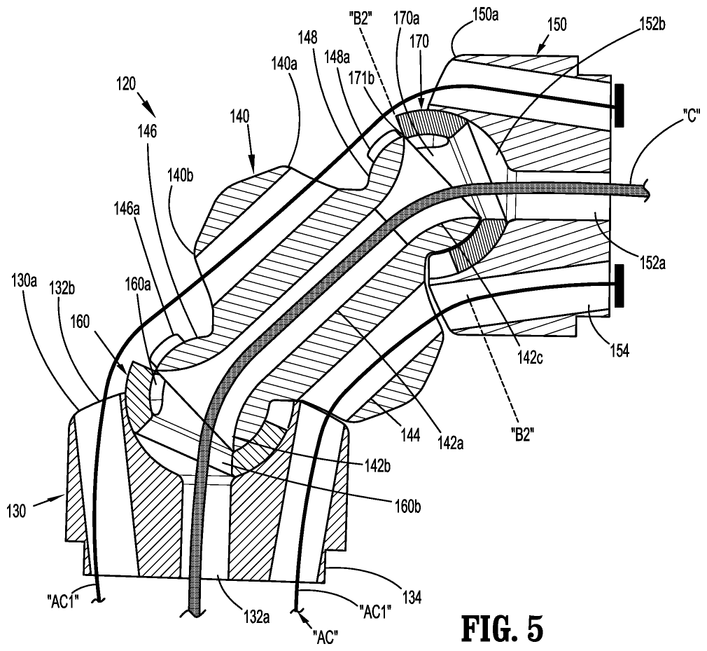

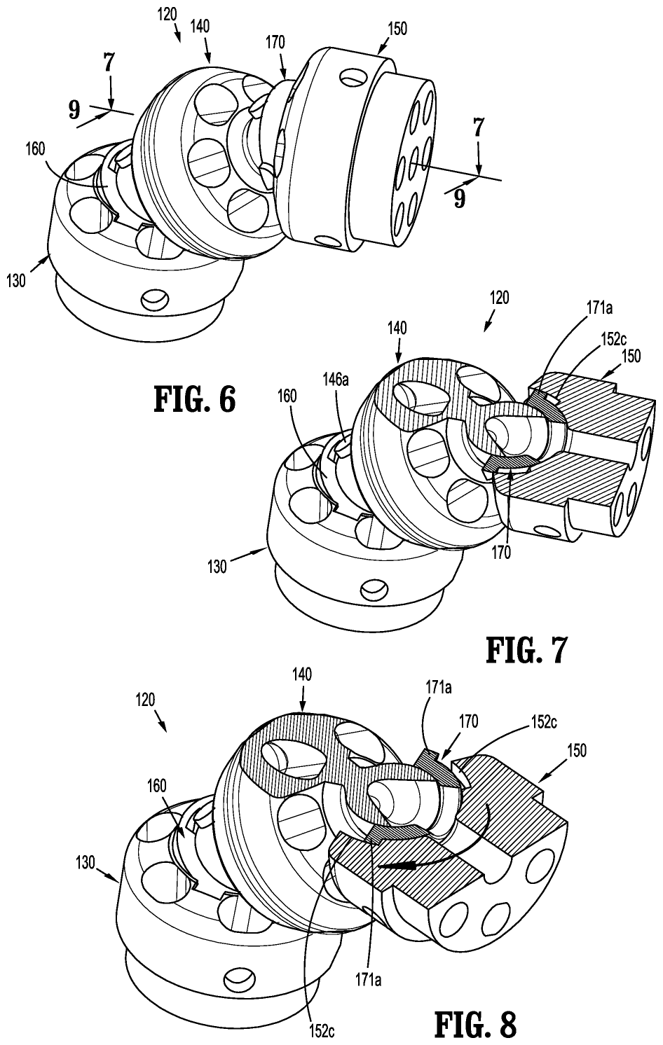

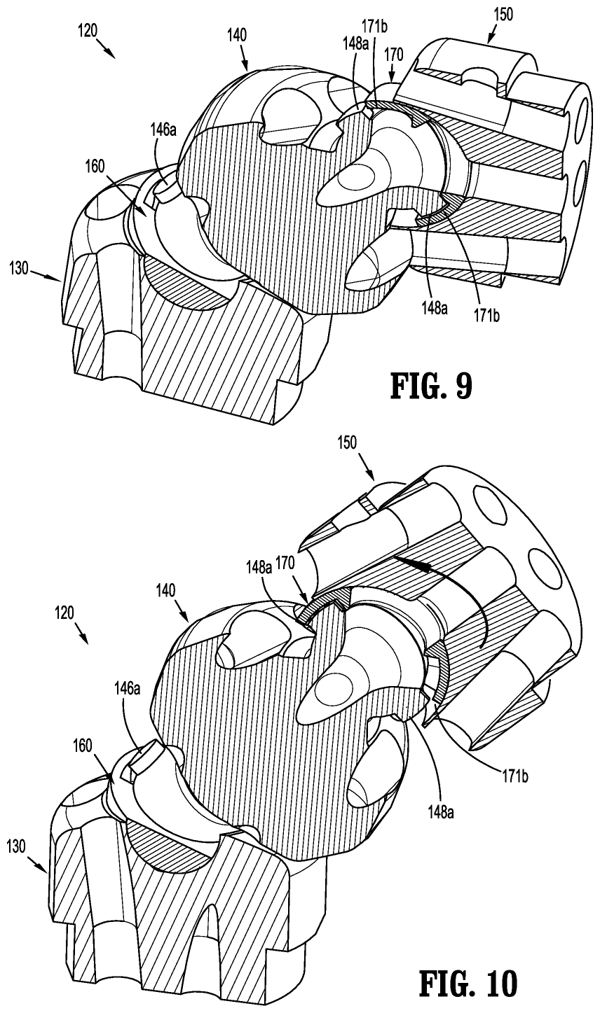

Turning now to FIGS. 2-10, surgical instrument 30 includes an elongate shaft 32 supporting the end effector 100 on a distal end thereof. Shaft 32 defines a longitudinal axis “X-X” about which end effector 100 can rotate. End effector 100 includes a jaw assembly 110 having a top or first jaw member 112, and a bottom or second jaw member 114 coupled to first jaw member 112. Jaw assembly 110 is connected to the distal end of shaft 32 by a wrist assembly 120. Jaw assembly 110 is positioned at a distal end of the wrist assembly 120 so as to be articulated relative to longitudinal axis “X-X” upon actuation of wrist assembly 120, as described below. Jaw assembly 110 of end effector 100 is coupled to one or more central cables, push/pull rods, etc. “C” (see FIG. 5), or the like, of surgical instrument 30 that are robotically actuatable to impart actuation (e.g., opening/closing movement) to jaw assembly 110.

Wrist assembly 120 includes several joint members pivotably interconnected with one another in tip-to-tail fashion. Specifically, wrist assembly 120 includes a proximal joint member 130, a middle or intermediate joint member 140 pivotably connected to proximal joint member 130, and a distal joint member 150 pivotably connected to middle joint member 140.

Proximal joint member 130 is non-rotatably supported on or at a distal end of elongate shaft 32. Proximal joint member 130 has a substantially cylindrical profile and defines a tapered or convex distal facing surface 130a. Proximal joint member 130 defines a central lumen 132a extending completely therethrough and being co-axial with a central longitudinal axis thereof. Central lumen 132a of proximal joint member 130 defines a distally facing concavity 132b formed at a distal end thereof. Proximal joint member 130 further includes a pair of diametrically opposed, radially inward extending, pivot grooves 132c formed along a rim of concavity 132b. Pivot grooves 132c define a first pivot axis “A1”.

Proximal joint member 130 further defines a plurality of longitudinally extending, radial lumens 134 disposed about central lumen 132a. Radial lumens 134 extend completely through proximal joint member 130, and may extend parallel to, or at an angle relative to, the central longitudinal axis of central lumen 132a thereof. Specifically, proximal joint member 130 defines an annular array of eight (8) radial lumens 134. While eight (8) radial lumens 134 are shown and described, it is contemplated that proximal joint member 130 may include fewer or more than eight (8) lumens 134.

Wrist assembly 120 includes a proximal spacer or bearing 160 slidably seated or nested in concavity 132b of proximal joint member 130. Bearing 160 is substantially hemispherical in shape and defines a distally facing concavity 160a and a central lumen 160b extending therethrough. Bearing 160 includes a pair of diametrically opposed radially outward projecting bosses 161a which are configured and dimensioned for pivotable seating in pivot grooves 132c of proximal joint member 130. A central axis of bosses 161a of bearing 160 is co-linear with first pivot axis “A1” of pivot grooves 132c of proximal joint member 130. Bearing 160 further includes a pair of diametrically opposed, radially inward extending, pivot grooves 161b formed along a rim of concavity 160a. Pivot grooves 161b define a second pivot axis “A2” which is orthogonal to first pivot axis “A1”.

Middle joint member 140 is non-rotatably connected to proximal joint member 130. Middle joint member 140 has a substantially cylindrical profile and defines a tapered or convex distal facing surface 140a, and a tapered or convex proximal facing surface 140b. Middle joint member 140 defines a central lumen 142a extending completely therethrough and being co-axial with a central longitudinal axis thereof, and with central lumen 132a of proximal joint member 130.

Middle joint member 140 further defines a plurality of longitudinally extending, radial lumens 144 disposed about central lumen 142a. Radial lumens 144 extend completely through middle joint member 140, and may extend parallel to, or at an angle relative to, the central longitudinal axis of central lumen 142a thereof. Specifically, middle joint member 140 defines an annular array of eight (8) radial lumens 144. While eight (8) radial lumens 144 are shown and described, it is contemplated that middle joint member 140 may include fewer or more than eight (8) lumens 144.

Middle joint member 140 includes a proximal ball connector 146 projecting from proximal facing surface 140b thereof. Proximal ball connector 146 is configured and dimensioned to be rotatably and slidably nested or seated in distally facing concavity 160a of bearing 160. It is contemplated that proximal ball connector 146 and concavity 160a of bearing 160 may be configured and dimensioned so as to form a snap-fit ball-socket connection therebetween. Proximal ball connector 146 includes a pair of diametrically opposed radially outward projecting bosses 146a which are configured and dimensioned for pivotable seating in pivot grooves 161b of bearing 160.

Middle joint member 140 includes a distal ball connector 148 projecting from distal facing surface 140a thereof. Distal ball connector 148 is configured and dimensioned to be rotatably and slidably nested or seated in a proximally facing concavity 170a of a bearing 170, as described below. It is contemplated that distal ball connector 148 and concavity 170a of bearing 170 may be configured and dimensioned so as to form a snap-fit ball-socket connection therebetween. Distal ball connector 148 includes a pair of diametrically opposed radially outward projecting bosses 148a which are configured and dimensioned for pivotable seating in pivot grooves 171b of bearing 170, as described below.

In an embodiment, a central axis of bosses 146a of proximal ball connector 146 and a central axis of bosses 148a of distal ball connector 148 are parallel with one another. While bosses 146a and bosses 148a are shown and described as being parallel, parallelity of bosses 146a, 148a is not required for functionality since both joints are satisfying the conical/spherical motion independently. However, parallel alignment of bosses 146a, 148a is highly practical giving the middle joint member symmetrical shape and helps to create better alignment to run the driving cables as well as other practical benefits.

It is contemplated that central lumen 142a of middle joint member 140 extends completely through proximal ball connector 146 and distal ball connector 148. It is further contemplated that a proximal end portion 142b of central lumen 142a flares radially outward in a conical or frusto-conical profile, and a distal end portion 142c of central lumen 142a also flares radially outward in a conical or frusto-conical profile.

Wrist assembly 120 includes a distal spacer or bearing 170 slidably seated or nested in a concavity 152b of distal joint member 150, described below. Bearing 170 is substantially hemispherical in shape and defines a proximally facing concavity 170a and a central lumen 170b extending therethrough. As described above, proximally facing concavity 170a of bearing 170 and distal ball connector 148 may be configured and dimensioned so as to form a snap-fit ball-socket connection therebetween. Bearing 170 includes a pair of diametrically opposed radially outward projecting bosses 171a which are configured and dimensioned for pivotable seating in pivot grooves 152c of distal joint member 150, described below. A central axis of bosses 171a of bearing 170 is co-linear with a first pivot axis “B1” of pivot grooves 152c of distal joint member 150, described below. Bearing 170 further includes a pair of diametrically opposed, radially inward extending, pivot grooves 171b formed along a rim of concavity 170a, and which are configured to pivotably receive bosses 148a of distal ball connector 148 of middle joint member 140. Pivot grooves 171b define a second pivot axis “B2” which is orthogonal to first pivot axis “B1” helps to maintain conical/spherical (wristed) motion for the ball joint connection.

Distal joint member 150 non-rotatably supports jaw assembly 110. Distal joint member 150 has a substantially cylindrical profile and defines a tapered or convex proximal facing surface 150a. Distal joint member 150 defines a central lumen 152a extending completely therethrough and being co-axial with a central longitudinal axis thereof. Central lumen 152a of distal joint member 150 defines a proximally facing concavity 152b formed at a proximal end thereof. Distal joint member 150 further includes a pair of diametrically opposed, radially inward extending, pivot grooves 152c formed along a rim of concavity 132b. Pivot grooves 132c define a pivot axis which is coaxial with pivot axis “B1” of central axis of bosses 171a of bearing 170.

Distal joint member 150 further defines a plurality of longitudinally extending, radial lumens 154 disposed about central lumen 152a. Radial lumens 154 extend completely through distal joint member 150, and may extend parallel to, or at an angle relative to, the central longitudinal axis of central lumen 152a thereof. Specifically, distal joint member 150 defines an annular array of four (4) radial lumens 154. While four (4) radial lumens 154 are shown and described, it is contemplated that distal joint member 150 may include fewer or more than four (4) lumens 154.

It is contemplated that first pivot axis “A1” of pivot grooves 132c of proximal joint member 130 and first pivot axis “B1” of central axis of bosses 171a of bearing 170 are in a common plane. It is further contemplated that second pivot axis “A2” of pivot grooves 161b of proximal bearing 160 and second pivot axis “B2” of pivot grooves 171b of bearing 170 are in a common plane.

It is also contemplated that a first pair of diametrically opposed radial lumens 154 of distal joint member 150 are aligned with first pivot axis “B1” of central axis of bosses 171a of bearing 170, and a second pair of diametrically opposed radial lumens 154 of distal joint member 150 are aligned with second pivot axis “B2” of pivot grooves 171b of bearing 170. It is further contemplated that the four (4) radial lumens 154 of distal joint member 150 are in registration with a corresponding four (4) radial lumens 144 of middle joint member 140. A remaining four (4) radial lumens 144 of the eight (8) total radial lumens 144 of middle joint member 140 are interposed between the first four (4) radial lumens 244. The eight (8) total radial lumens 144 of middle joint member 140 are in registration with the eight (8) radial lumens 134 of proximal joint member 130.

Surgical instrument 30 includes either (8) articulation cables “AC” extending through elongate shaft 32. Articulation cables “AC” each have a proximal end (not shown) which are operatively engaged with motors of robotic arm 20 and/or robotic surgical system.

Four (4) articulation cables “AC1” of the eight (8) articulation cables “AC” include distal ends disposed within or extending through radial lumens 154 of distal joint member 150, wherein these distal ends of articulation cables “AC1” are anchored or otherwise secured to distal joint member 150. The articulation cables “AC1” extend proximally from distal joint member 150, through corresponding radial lumens 144 of middle joint member 140, and proximally through corresponding radial lumens 134 of proximal joint member 130.

A remaining four (4) articulation cables “AC2” of the eight (8) articulation cables “AC” include distal ends disposed within or extending through the non-occupied radial lumens 144 of middle joint member 140, wherein these distal ends of articulation cables “AC2” are anchored or otherwise secured to middle joint member 140. The articulation cables “AC2” extend proximally from middle joint member 140, through corresponding radial lumens 134 of proximal joint member 130.

It is contemplated that joint members 130, 140, 150, and bearings 160, 170 are fabricated from stainless steel, titanium, polymers, resins, composite materials, nylons, silicones and the like. In an embodiment, joint members 130, 140, 150 are fabricated from stainless steel and bearings 160, 170 are fabricated from nylon (or other lubricious material).

In accordance with the disclosure, it is contemplated that proximal retraction of at least one of articulation cables “AC” will result in articulation of wrist assembly 120 of surgical instrument 30. It is contemplated that wrist assembly 120 may be articulated in multiple directions depending on the coordinated proximal retractions of articulation cables “AC”. It is further contemplated that as one or more articulation cables “AC” are retracted proximally, at least one or more articulation cables “AC” must be permitted to translate distally.

Wrist assembly 120 defines a first degree of freedom between proximal joint member 130 and middle joint member 140 (along the first pivot axis “A1” defined by pivot grooves 132c formed along concavity 132b of proximal joint member 130), a second degree of freedom between proximal joint member 130 and middle joint member 140 (along the second pivot axis “A2” defined by pivot grooves 160a formed along concavity 160a of proximal bearing 160), a third degree of freedom between middle joint member 140 and distal joint member 150 (along the first pivot axis “B1” defined by pivot grooves 152c formed along concavity 152b of distal joint member 150), and a fourth degree of freedom between middle joint member 140 and distal joint member 150 (along the second pivot axis “B2” defined by pivot grooves 171b formed along concavity 170a of distal bearing 170).

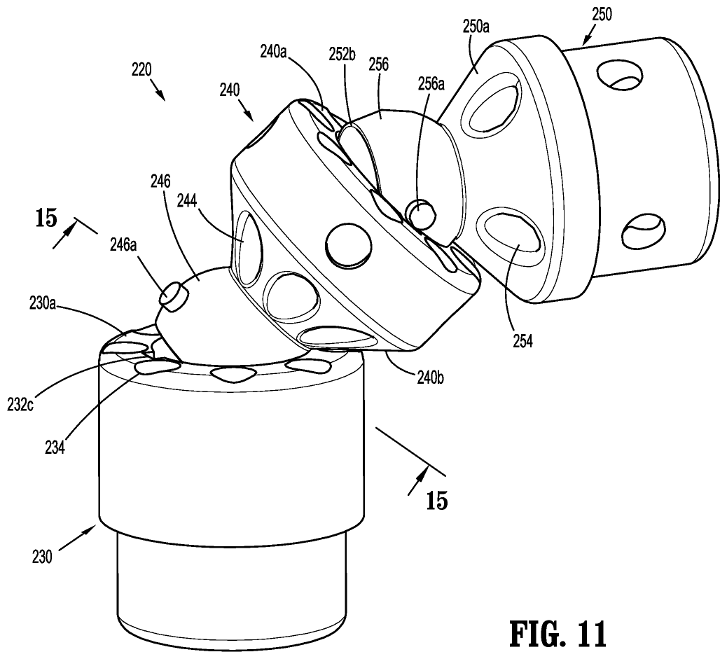

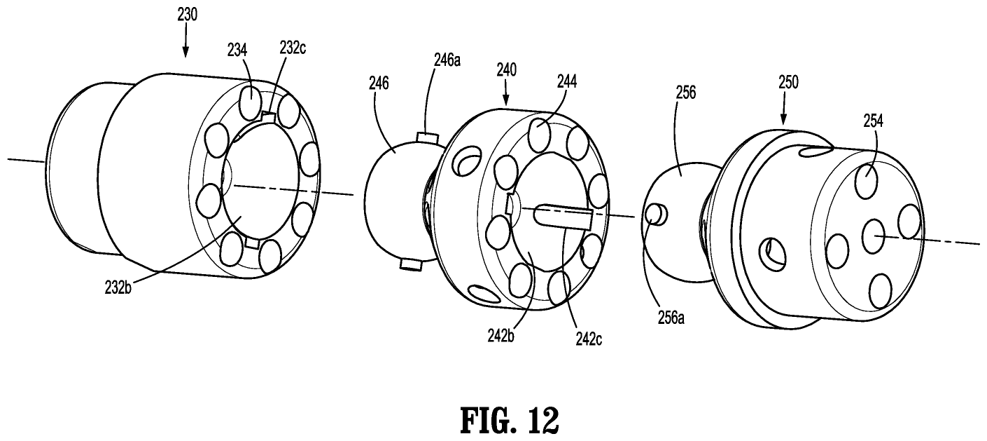

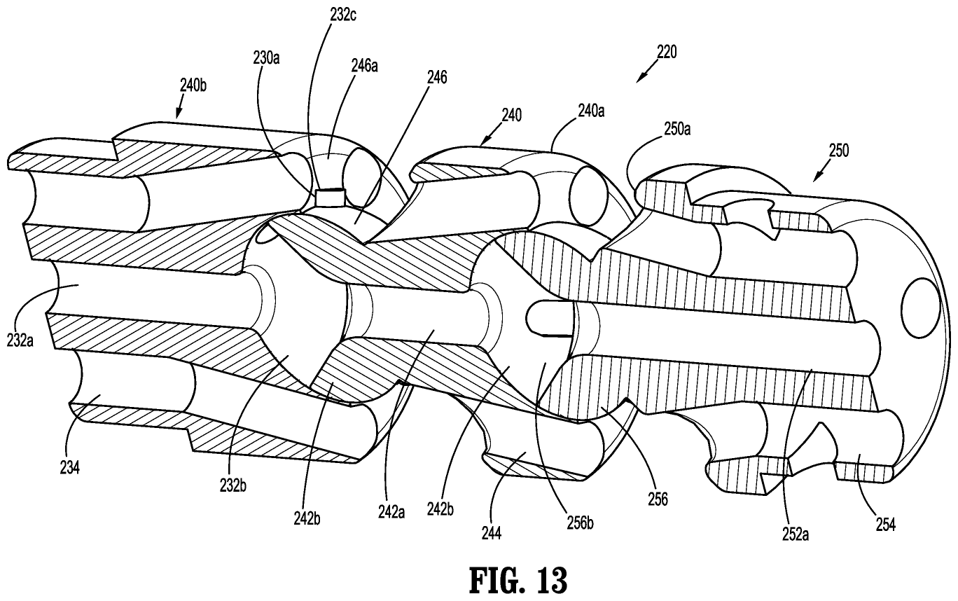

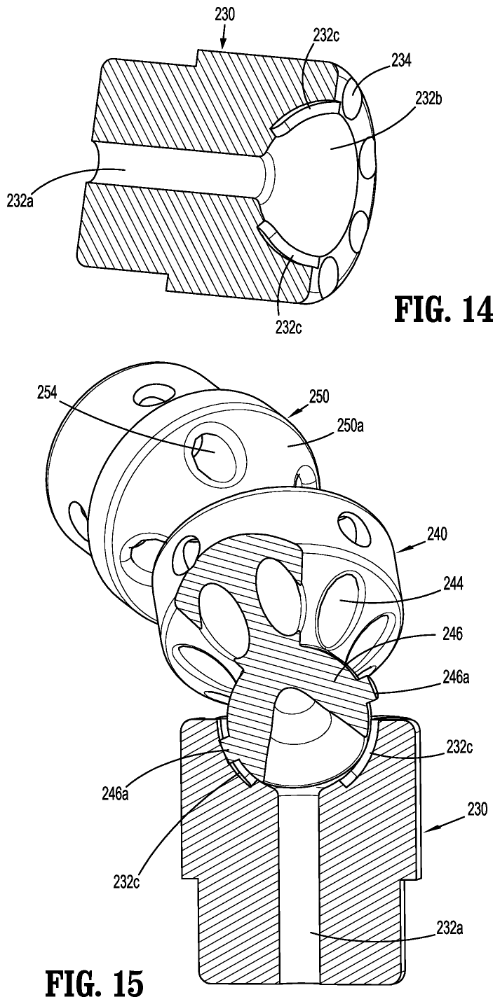

Turning now to FIGS. 11-15, a wrist assembly 220 according to another embodiment of the disclosure, for incorporation into a surgical instrument (e.g., surgical instrument 30), is shown. Wrist assembly 220 includes several joint members pivotably interconnected with one another in tip-to-tail fashion. Specifically, wrist assembly 220 includes a proximal joint member 230, a middle or intermediate joint member 240 pivotably connected to proximal joint member 230, and a distal joint member 250 pivotably connected to middle joint member 240.

Proximal joint member 230 is non-rotatably supported on or at a distal end of the elongate shaft of the surgical instrument. Proximal joint member 230 has a substantially cylindrical profile and defines a planar distal facing surface 230a. Proximal joint member 230 defines a central lumen 232a extending completely therethrough and being co-axial with a central longitudinal axis thereof. Central lumen 232a of proximal joint member 230 defines a distally facing concavity 232b formed at a distal end thereof. Proximal joint member 230 further includes a pair of diametrically opposed, radially inward extending, pivot grooves 232c formed along a rim of concavity 232b. Pivot grooves 232c define a first pivot axis.

Proximal joint member 230 further defines a plurality of longitudinally extending, radial lumens 234 disposed about central lumen 232a. Radial lumens 234 extend completely through proximal joint member 230, and may extend parallel to, and/or at an angle relative to, the central longitudinal axis of central lumen 232a thereof. Specifically, proximal joint member 230 defines an annular array of eight (8) radial lumens 234. While eight (8) radial lumens 234 are shown and described, it is contemplated that proximal joint member 230 may include fewer or more than eight (8) lumens 234.

Middle joint member 240 is non-rotatably connected to proximal joint member 230. Middle joint member 240 has a substantially cylindrical profile and defines a planar distal facing surface 240a, and a tapered or conical proximal facing surface 240b. Middle joint member 240 defines a central lumen 242a extending completely therethrough and being co-axial with a central longitudinal axis thereof, and with central lumen 232a of proximal joint member 230.

Middle joint member 240 further defines a plurality of longitudinally extending, radial lumens 244 disposed about central lumen 242a. Radial lumens 244 extend completely through middle joint member 240, and may extend parallel to, and/or at an angle relative to, the central longitudinal axis of central lumen 242a thereof. Specifically, middle joint member 240 defines an annular array of eight (8) radial lumens 244. While eight (8) radial lumens 244 are shown and described, it is contemplated that middle joint member 240 may include fewer or more than eight (8) lumens 244.

Middle joint member 240 includes a proximal ball connector 246 projecting from proximal facing surface 240b thereof. Proximal ball connector 246 is configured and dimensioned to be rotatably and slidably nested or seated in distally facing concavity 232b of proximal joint member 230. It is contemplated that proximal ball connector 246 and concavity 232b of proximal joint member 230 may be configured and dimensioned so as to form a snap-fit ball-socket connection therebetween. Proximal ball connector 246 includes a pair of diametrically opposed radially outward projecting bosses 246a which are configured and dimensioned for pivotable seating in pivot grooves 232c of proximal joint member 230.

Middle joint member 240 defines a distally facing concavity 242b formed at a distal end thereof. Middle joint member 240 further includes a pair of diametrically opposed, radially inward extending, pivot grooves 242c formed along a rim of concavity 242b. Pivot grooves 242c define a second pivot axis. The second pivot axis of pivot grooves 242c of middle joint member 240 is oriented orthogonally to the first pivot axis of pivot grooves 232c of proximal joint member 230.

It is contemplated that central lumen 242a of middle joint member 240 extends completely through proximal ball connector 246 and distally facing concavity 242b. It is further contemplated that a proximal end portion 242b of central lumen 242a flares radially outward in a conical or frusto-conical profile.

Distal joint member 250 non-rotatably supports jaw assembly 110. Distal joint member 250 has a substantially cylindrical profile and defines a tapered or conical proximal facing surface 250a. Distal joint member 250 defines a central lumen 252a extending completely therethrough and being co-axial with a central longitudinal axis thereof.

Distal joint member 250 further defines a plurality of longitudinally extending, radial lumens 254 disposed about central lumen 252a. Radial lumens 254 extend completely through distal joint member 250, and may extend parallel to, and/or at an angle relative to, the central longitudinal axis of central lumen 252a thereof. Specifically, distal joint member 250 defines an annular array of four (4) radial lumens 154. While four (4) radial lumens 254 are shown and described, it is contemplated that distal joint member 250 may include fewer or more than four (4) lumens 254.

Distal joint member 250 includes a proximal ball connector 256 projecting from proximal facing surface 250b thereof. Proximal ball connector 256 is configured and dimensioned to be rotatably and slidably nested or seated in distally facing concavity 242b of middle joint member 240. It is contemplated that proximal ball connector 256 of distal joint member 250 and concavity 242b of middle joint member 240 may be configured and dimensioned so as to form a snap-fit ball-socket connection therebetween. Proximal ball connector 256 includes a pair of diametrically opposed radially outward projecting bosses 256a which are configured and dimensioned for pivotable seating in pivot grooves 242c of middle joint member 240.

It is contemplated that a first pair of diametrically opposed radial lumens 254 of distal joint member 250 are aligned with the second pivot axis of pivot grooves 242c of middle joint member 240, and a second pair of diametrically opposed radial lumens 254 of distal joint member 250 are aligned with the first pivot axis of pivot grooves 232c of proximal joint member 230.

It is further contemplated that the four (4) radial lumens 254 of distal joint member 250 are in registration with a corresponding four (4) radial lumens 244 of middle joint member 240. A remaining four (4) radial lumens 244 of the eight (8) total radial lumens 244 of middle joint member 240 are interposed between the first four (4) radial lumens 244. The eight (8) total radial lumens 244 of middle joint member 240 are in registration with the eight (8) radial lumens 234 of proximal joint member 230.

Wrist assembly 220 may include a proximal spacer or bearing and/or distal spacer or bearing (not shown), in the form of proximal spacer bearing 160 or distal spacer bearing 170 of wrist assembly 120, described above.

Similar to a surgical instrument 30 including a wrist assembly 120, a surgical instrument including a wrist assembly 230 includes either (8) articulation cables “AC” extending through elongate shaft 32 and operatively associated with respective middle joint member 240 and distal joint member 250.

Wrist assembly 220 defines a first degree of freedom between proximal joint member 230 and middle joint member 240 (along the first pivot axis defined by pivot grooves 232c formed along concavity 232b of proximal joint member 230), and a second degree of freedom between middle joint member 240 and distal joint member 250 (along the second pivot axis defined by pivot grooves 242c formed along concavity 242b of middle joint member 240).

Proximal joint member 230 further includes a pair of diametrically opposed, radially inward extending, pivot grooves 232c formed along a rim of concavity 232b. Pivot grooves 232c

Persons skilled in the art will understand that the structures and methods specifically described herein and illustrated in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, it is envisioned that the elements and features illustrated or described in connection with one exemplary embodiment may be combined with the elements and features of another without departing from the scope of the present disclosure, and that such modifications and variations are also intended to be included within the scope of the present disclosure. Indeed, any combination of any of the presently disclosed elements and features is within the scope of the present disclosure. Accordingly, the subject matter of the present disclosure is not to be limited by what has been particularly shown and described.

1. A surgical instrument for a robotic surgical system, the surgical instrument comprising:
an elongate shaft defining a longitudinal axis;
an end effector having a first jaw member and a second jaw member movably coupled to one another; and
a wrist assembly interconnecting the elongate shaft and the end effector, the wrist assembly permitting a longitudinal axis of the end effector to be rotated or translated relative to the longitudinal axis of the elongate shaft, the wrist assembly including:
a proximal joint member supported on a distal end of the elongate shaft, the proximal joint member defines:
a longitudinally extending central lumen;
a plurality of longitudinally extending radial lumens arranged in a radial array around the central lumen thereof; and
a concavity formed in a distal facing surface thereof, wherein the concavity of the proximal joint member is centrally aligned with the central lumen thereof;
a distal joint member supported on a proximal end of the end effector, the distal joint member defines:
a longitudinally extending central lumen;
a plurality of longitudinally extending radial lumens arranged in a radial array around the central lumen thereof; and
a concavity formed in a proximal facing surface thereof, wherein the concavity of the distal joint member is centrally aligned with the central lumen thereof; and
a middle joint member interposed between the proximal joint member and the distal joint member, the middle joint member includes a proximal ball connector extending proximally therefrom and a distal ball connector extending distally therefrom, wherein the proximal ball connector is seated within the concavity formed in the distal facing surface of the proximal joint member and the distal ball connector is seated within the concavity formed in the proximal facing surface of the proximal joint member, the distal joint member defines:
a longitudinally extending central lumen; and
a plurality of longitudinally extending radial lumens arranged in a radial array around the central lumen thereof.

2. The surgical instrument of claim 1, wherein the wrist assembly includes:
a proximal bearing disposed within the concavity of the proximal joint member, and interposed between the proximal joint member and the proximal ball connector of the middle joint member; and
a distal bearing disposed within the concavity of the distal joint member, and interposed between the distal joint member and the distal ball connector of the middle joint member.

3. The surgical instrument of claim 2, wherein:
the proximal bearing defines a pair of diametrically opposed, radially outwardly projecting bosses that define a pivot axis therethrough; and
the distal bearing defines a pair of diametrically opposed, radially outwardly projecting bosses that define a pivot axis therethrough.

4. The surgical instrument of claim 3, wherein the pivot axis of the pair of bosses of the proximal bearing and the pivot axis of the pair of bosses of the distal bearing reside in a common plane.

5. The surgical instrument of claim 4, wherein:
the proximal joint member defines a pair of diametrically opposed, radially outward projecting pivot grooves formed in a rim of the concavity thereof and being configured and dimensioned to pivotably receive the pair of bosses of the proximal bearing; and
the distal joint member defines a pair of diametrically opposed, radially outward projecting pivot grooves formed in a rim of the concavity thereof and being configured and dimensioned to pivotably receive the pair of bosses of the distal bearing.

6. The surgical instrument of claim 5, wherein:
the proximal ball connector of the middle joint member defines a pair of diametrically opposed, radially outwardly projecting bosses that define a pivot axis therethrough; and
the distal ball connector of the middle joint member defines a pair of diametrically opposed, radially outwardly projecting bosses that define a pivot axis therethrough.

7-9. (canceled)

10. The surgical instrument of claim 6, wherein:
the proximal bearing defines a pair of diametrically opposed, radially inward projecting pivot grooves formed in a rim of a concavity thereof and which grooves are configured and dimensioned to pivotably receive the pair of bosses of the proximal ball connector of the middle joint member; and
the distal bearing defines a pair of diametrically opposed, radially inward projecting pivot grooves formed in a rim of a concavity thereof and which grooves are configured and dimensioned to pivotably and slidably receive the pair of bosses of the distal ball connector of the middle joint member.

11. The surgical instrument of claim 10, wherein:
the distal joint member includes four longitudinally extending radial lumens arranged in a radial array around the central lumen thereof; and
the middle joint member includes eight longitudinally extending radial lumens arranged in a radial array around the central lumen thereof, wherein four of the eight radial lumens of the middle joint member are in registration with the four radial lumens of the distal joint member.

12. The surgical instrument of claim 11, wherein:
the proximal joint member includes eight longitudinally extending radial lumens arranged in a radial array around the central lumen thereof, wherein the eight radial lumens of the proximal joint member are in registration with the eight radial lumens of the middle joint member.

13. The surgical instrument of claim 12, further comprising a plurality of articulation cables extending through the wrist assembly, wherein:
four articulation cables of the plurality of cables include distal ends secured to the distal joint member, and proximal ends slidably extending through the four longitudinally extending radial lumens of the middle joint member that are in registration with the four longitudinally extending radial lumens of the distal joint member; and
four articulation cables of the plurality of cables include distal ends secured to the middle member, and proximal ends slidably extending through the four longitudinally extending radial lumens of the proximal joint member that are in registration with the four un-occupied longitudinally extending radial lumens of the middle joint member.

14. (canceled)

15. A wrist assembly for a surgical instrument, the wrist assembly comprising:
a proximal joint member configured for support on a distal end of an elongate shaft, the proximal joint member defines:
a longitudinally extending central lumen;
a plurality of longitudinally extending radial lumens arranged in a radial array around the central lumen thereof; and
a concavity formed in a distal facing surface thereof, wherein the concavity of the proximal joint member is centrally aligned with the central lumen thereof;
a distal joint member configured to support a proximal end of a surgical end effector, the distal joint member defines:
a longitudinally extending central lumen;
a plurality of longitudinally extending radial lumens arranged in a radial array around the central lumen thereof; and
a concavity formed in a proximal facing surface thereof, wherein the concavity of the distal joint member is centrally aligned with the central lumen thereof; and
a middle joint member interposed between the proximal joint member and the distal joint member, the middle joint member includes a proximal ball connector extending proximally therefrom and a distal ball connector extending distally therefrom, wherein the proximal ball connector is seated within the concavity formed in the distal facing surface of the proximal joint member and the distal ball connector is seated within the concavity formed in the proximal facing surface of the proximal joint member, the distal joint member defines:
a longitudinally extending central lumen; and
a plurality of longitudinally extending radial lumens arranged in a radial array around the central lumen thereof.

16. The wrist assembly of claim 15, further comprising:
a proximal bearing disposed within the concavity of the proximal joint member, and interposed between the proximal joint member and the proximal ball connector of the middle joint member; and
a distal bearing disposed within the concavity of the distal joint member, and interposed between the distal joint member and the distal ball connector of the middle joint member.

17. The wrist assembly of claim 16, wherein:
the proximal bearing defines a pair of diametrically opposed, radially outwardly projecting bosses that define a pivot axis therethrough; and
the distal bearing defines a pair of diametrically opposed, radially outwardly projecting bosses that define a pivot axis therethrough.

18. The wrist assembly of claim 17, wherein the pivot axis of the pair of bosses of the proximal bearing and the pivot axis of the pair of bosses of the distal bearing reside in a common plane.

19. The wrist assembly of claim 18, wherein:
the proximal joint member defines a pair of diametrically opposed, radially outward projecting pivot grooves formed in a rim of the concavity thereof and being configured and dimensioned to pivotably and slidably receive the pair of bosses of the proximal bearing; and
the distal joint member defines a pair of diametrically opposed, radially outward projecting pivot grooves formed in a rim of the concavity thereof and being configured and dimensioned to pivotably and slidably receive the pair of bosses of the distal bearing.

20. The wrist assembly of claim 19, wherein:
the proximal ball connector of the middle joint member defines a pair of diametrically opposed, radially outwardly projecting bosses that define a pivot axis therethrough; and
the distal ball connector of the middle joint member defines a pair of diametrically opposed, radially outwardly projecting bosses that define a pivot axis therethrough.

21-23. (canceled)

24. The wrist assembly of claim 20, wherein:
the proximal bearing defines a pair of diametrically opposed, radially inward projecting pivot grooves formed in a rim of a concavity thereof and which grooves are configured and dimensioned to pivotably and slidably receive the pair of bosses of the proximal ball connector of the middle joint member; and
the distal bearing defines a pair of diametrically opposed, radially inward projecting pivot grooves formed in a rim of a concavity thereof and which grooves are configured and dimensioned to pivotably and slidably receive the pair of bosses of the distal ball connector of the middle joint member.

25. The wrist assembly of claim 24, wherein:
the distal joint member includes four longitudinally extending radial lumens arranged in a radial array around the central lumen thereof; and
the middle joint member includes eight longitudinally extending radial lumens arranged in a radial array around the central lumen thereof, wherein four of the eight radial lumens of the middle joint member are in registration with the four radial lumens of the distal joint member.

26. The wrist assembly of claim 25, wherein:
the proximal joint member includes eight longitudinally extending radial lumens arranged in a radial array around the central lumen thereof, wherein the eight radial lumens of the proximal joint member are in registration with the eight radial lumens of the middle joint member.

27. The wrist assembly of claim 26, further comprising a plurality of articulation cables extending therethrough, wherein:
four articulation cables of the plurality of cables include distal ends secured to the distal joint member, and proximal ends slidably extending through the four longitudinally extending radial lumens of the middle joint member that are in registration with the four longitudinally extending radial lumens of the distal joint member; and
four articulation cables of the plurality of cables include distal ends secured to the middle member, and proximal ends slidably extending through the four longitudinally extending radial lumens of the proximal joint member that are in registration with the four un-occupied longitudinally extending radial lumens of the middle joint member.

28. (canceled)
